(19) United States
(12) Patent Application Publication
(10) Pub. No.: US 2026/0013825 A1
(43) Pub. Date: Jan. 15, 2026

(54) FLOW VARIATION ANALYSIS OF LOWER EXTREMITIES USING ULTRASOUND BLOOD FLOW IMAGING

(71) Applicant: Mayo Foundation for Medical Education and Research, Rochester, MN (US)

(72) Inventors: Azra Alizad, Rochester, MN (US); Mostafa Fatemi, Rochester, MN (US); Rohit Nayak, Rochester, NY (US); Soroosh Sabeti, Rochester, MN (US)

(21) Appl. No.: 19/265,573

(22) Filed: Jul. 10, 2025

Related U.S. Application Data
(60) Provisional application No. 63/670,586, filed on Jul. 12, 2024.

(51) Int. Cl. A61B 8/06; A61B 8/00; G16H 50/20; G16H 50/30; G16H 50/70

(52) U.S. Cl. CPC A61B 8/06; A61B 8/488; A61B 8/5223; G16H 50/20; G16H 50/30; G16H 50/70

(57) ABSTRACT

Blood flow in the lower extremities is assessed using ultrasound. A pressure cuff is wrapped around the lower extremity and rapidly inflated and deflated. Ultrasound data are acquired before, during, and after the compression of the lower extremity. Doppler image frames are generated from the ultrasound data, and correlation map data are generated by correlating the Doppler image frames with one or more activation functions that each model the compression applied to the lower extremity. Flow variation metric data are generated from the correlation map data and can be outputted using a computer system.

### CROSS-REFERENCE TO RELATED APPLICATIONS

This application claims the benefit of U.S. Provisional Patent Application Ser. No. 63/670,586, filed on Jul. 12, 2024, and entitled “FLOW VARIATION ANALYSIS OF LOWER EXTREMITIES USING ULTRASOUND BLOOD FLOW IMAGING,” which is herein incorporated by reference in its entirety.

### STATEMENT OF FEDERALLY SPONSORED RESEARCH

This invention was made with government support under HL148664 awarded by the National Institutes of Health. The government has certain rights in the invention.

### BACKGROUND

Patients with peripheral arterial disease (PAD) usually suffer from impairments of the circulatory functions in the lower limb, leading to inadequate blood supply. Monitoring the variations in blood flow and perfusion within leg muscles can provide diagnostic information regarding the state of the disease. Common clinical measures for PAD diagnosis can either lack accuracy or come with concerns in terms of invasiveness, availability, and cost.

### SUMMARY OF THE DISCLOSURE

It is an aspect of the present disclosure to provide a method for generating quantitative flow variation metrics from non-contrast ultrasound data. The method includes providing ultrasound data to a computer system, where the ultrasound data have been acquired with an ultrasound system from a lower extremity of a subject. The ultrasound data are acquired during three durations of time: a first duration of time during which no compression is applied to the lower extremity; a second duration of time during which compression is applied to the lower extremity, where the second duration of time occurs after the first duration of time; and a third duration of time during which no compression is applied to the lower extremity, where the third duration of time occurs after the second duration of time. The method also includes generating a series of Doppler image frames from the ultrasound data, where the Doppler image frames depict perfusion in the lower extremity of the subject. Correlation map data are generated with the computer system by correlating the series of Doppler image frames with at least one activation function that models the compression applied to the lower extremity. Flow variation metric data can then be generated from the correlation map data using the computer system, and the flow variation metric data may be outputted using the computer system. Other embodiments of this aspect include corresponding systems (e.g., computer systems), programs, algorithms, and/or modules, each configured to perform the steps of the methods.

According to another aspect of the present disclosure, a method for assessing blood flow in a lower extremity using ultrasound is provided. The method includes acquiring ultrasound data from the lower extremity of a subject wearing a pressure cuff around a portion of their lower extremity while the pressure cuff is applying a compression to the lower extremity. The method also includes generating one or more Doppler image frames from the ultrasound data, generating correlation map data from the Doppler image frames based on correlation with one or more activation functions that model the compression applied to the lower extremity by the pressure cuff, and generating flow variation metric data from the correlation map data, wherein the flow variation metric data provide a quantitative assessment of blood flow in the lower extremity.

According to another aspect of the present disclosure, a method for generating flow variation metrics from ultrasound data acquired from a lower extremity of a subject is provided. The method includes acquiring ultrasound data from the lower extremity of the subject wearing a pressure cuff around a portion of the lower extremity, generating one or more Doppler image frames from the ultrasound data, generating correlation maps from the Doppler image frames using a lag activation function, generating lag image data from the Doppler image frames, generating maximum correlation map data from the Doppler image frames, and generating flow variation metric data from the correlation maps, lag image data, and maximum correlation map data.

### BRIEF DESCRIPTION OF THE DRAWINGS

FIG. 1 is a flowchart of an example method for generating flow variation metrics from ultrasound data according to embodiments described in the present disclosure.

FIG. 2 illustrates an example experimental setup for data acquisition. An ultrasound probe is fixed using a mechanical arm and is attached to the calf muscle for the duration of the study. The leg is placed on a leg prepper and a pressure cuff is wrapped around the thigh.

FIG. 3 illustrates an example timeline of a data acquisition procedure, including a minute of baseline acquisition, three minutes of pressure cuff occlusion, and three minutes of post-occlusion acquisition. Example Doppler images are shown within each section of the timeline.

FIG. 4 shows an example workflow for Doppler image generation according to embodiments described in the present disclosure.

FIG. 5 an example workflow for hemodynamic response analysis according to embodiments described in the present disclosure.

FIG. 6 illustrates an example workflow for the processes according to some embodiments described in the present disclosure.

FIGS. 7A-7L illustrate a comparison of the results for the legs of a healthy individual (FIGS. 7A-7F) and a PAD patient with abnormal ABI (FIGS. 7G-7L). FIGS. 7A and 7G show B-modes of the scanned region. FIGS. 7B and 7H show Doppler images post-occlusion. FIGS. 7C and 7I show binarized post-occlusion correlation masks using a 0.5 threshold on the correlation value. FIGS. 7D and 7J show lag images. FIGS. 7E and 7K show normalized average of Doppler Intensity variations (red) of all pixels within correlation masks (shaded light blue region depicts half of the standard deviation on each side of the plot) and the single frame lag activation function (blue), for 10 samples leading to the pressure release point (PRP). FIGS. 7F and 7L show average Doppler intensity variations within the combined mask as a function of time (Doppler frames). Shaded light blue region shows half of the standard deviation on each side of the plot. Green and red circles denote the cuff inflation and deflation points, respectively.

FIGS. 8A-8D. illustrate box-and-whisker plots showing the distributions of the metrics. FIG. 8A shows post-occlusion to baseline flow intensity variation (PBFIV). FIG. 8B shows total response region (TRR). FIG. 8C shows lag0 response region (LORR). FIG. 8D shows lag4 (and more) response region (L4+RR).

FIG. 9 is a block diagram of an example system for performing hemodynamic response analysis according to embodiments described in the present disclosure.

FIG. 10 is a block diagram of example components that can implement the system of FIG. 9.

FIG. 11 is a block diagram of an example ultrasound system that can be implemented according to some embodiments described in the present disclosure.

### DETAILED DESCRIPTION

Described here are systems and methods for assessing blood flow in the lower extremities using ultrasound. A pressure cuff is wrapped around the calf muscle and rapidly inflated and deflated, then measuring the subsequent blood flow. A series of metrics are determined to quantify blood flow at the microvascular level, which can better assess peripheral artery disease (PAD) and other vascular conditions affecting the lower extremities.

Advantageously, no contrast agent is needed with this technique, making it less invasive than standard techniques that require use of contrast agents. As another advantage, the disclosed systems and methods are capable of capturing slower blood flow.

It is an aspect of the disclosed systems and methods to provide the ability to perform both fast imaging and contrast-free imaging. It is another aspect of the disclosed systems and method to provide the use of several metrics to quantitatively assess blood flow.

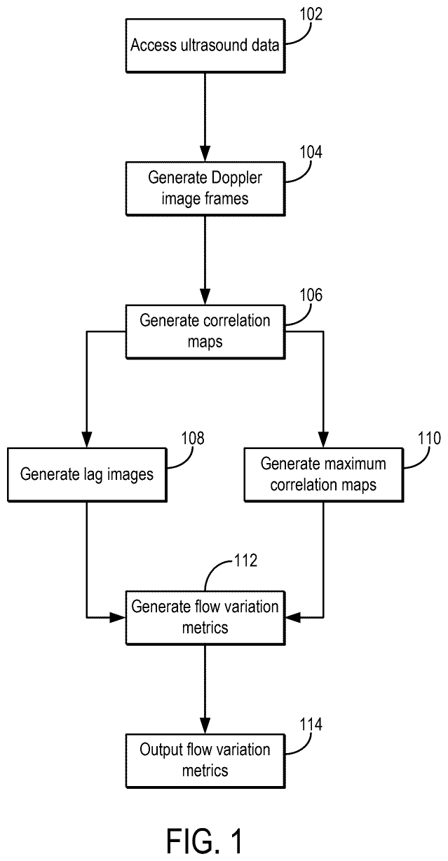

Referring now to FIG. 1, a flowchart is illustrated as setting forth the steps of an example method for generating flow variation metrics from ultrasound data acquired from a lower extremity of a subject.

The method includes accessing ultrasound data with a computer system, as indicated at step 102. Accessing the ultrasound data may include retrieving such data from a memory or other suitable data storage device or medium. Additionally or alternatively, accessing the ultrasound data may include acquiring such data with an ultrasound and transferring or otherwise communicating the data to the computer system, which may be a part of the ultrasound system.

In general, the ultrasound data are acquired from a subject wearing a pressure cuff around a portion of their lower extremity. For example, the ultrasound data may be acquired from a subject wearing a pressure cuff around their thigh or another portion of the lower extremity. The pressure cuff is operable to apply a pressure to the lower extremity during a duration of time in which the ultrasound data are acquired. The pressure applied to the lower extremity may be a constant pressure or a variable pressure. Additionally or alternatively, both constant and variable pressures may be applied to the lower extremity during the duration of time. In some instances, the pressure applied to the lower extremity is sufficient to completely occlude blood flow while the pressure is applied. Alternatively, the pressure may be sufficient to only partially occlude blood flow.

While the subject is lying down in the supine position, ultrasound data are acquired before, during, and after pressure is applied to the lower extremity by the pressure cuff. In this way, blood flow in the lower extremity can be constantly monitored over a duration of time in which different flow conditions are created in the lower extremity by the application of pressure (or multiple different pressures) to the lower extremity by the pressure cuff. As a non-limiting example, ultrasound data can be acquired over a seven minute duration of time.

The ultrasound data can include data acquired using high-frame-rate plane-wave ultrasound microvessel imaging without using contrast agents. As a non-limiting example, the ultrasound data can be acquired using a linear array with a center frequency of 7.24 MHz, or other suitable center frequency. Ultrafast ultrasound imaging can be implemented through coherent compounding of plane wave transmissions at multiple different insonification angles. For example, plane wave transmissions at five different insonification angles that are equally spaced within the [−5.5°+5.5°] range can be used. Each transmission sequence involved acquiring multiple IQ data frames (e.g., 500 or more IQ data frames). In a non-limiting example, the data frames were acquired at a frame rate of 2 kHz and the transmission sequence was repeated every 2 s to monitor the flow response over time.

One or more Doppler image frames, which may be referred to as perfusion images, are then generated from the ultrasound data, as indicated at step 104. The ultrasound data frames acquired during each transmission sequence constitute an ensemble of ultrasound images that can be used to generate a Doppler image visualizing blood flow intensities at a given point in time. These frames can first be reshaped into a spatiotemporal data matrix, such as a Casorati data matrix. Next, a singular value decomposition (SVD) filter, or other suitable clutter filter, can be applied to the data to remove the low-rank tissue clutter. In a non-limiting example, the SVD threshold used for the separation of clutter and blood subspaces was chosen empirically and was set to the constant value of 50 for all the Doppler frames (no upper threshold for noise removal). Subsequently, the clutter filtered data frame ensemble can undergo temporal coherent integration to generate the Doppler image.

The resulting Doppler image is composed of signals generated by blood flow as well as noise. The background noise profile, which may be induced by the time gain compensation (TGC) settings, can be estimated and compensated for. As one non-limiting example, a Doppler image generated from an open-air transmission can be used to estimate this background noise profile. Since no significant echo signal is expected in such a transmission, the resulting image can be a good approximation of the TGC-induced noise pattern. The background noise profile can then be subtracted from each of the Doppler images to generate a final flow image. The images may each be referred to as a Doppler frame.

The generated Doppler images, or frames, illustrate the hemodynamic variations in the lower extremity (e.g., the calf muscle). These variations can be quantified as a function of time in a time series. As one non-limiting example, the quantified function of time may have a sampling period of 2 s.

Correlation maps are then generated from the ultrasound data using a lag activation function, as indicated at step 106. To monitor the hyperemic response to the pressure induced occlusion, the lag activation step function is defined to represent the onset of the stimulation (pressure release) at the “deflate” (pressure release) point.

The correlation maps may include one or more correlation maps. A correlation map may be generated by correlating temporal Doppler signal intensity variations in the ultrasound data with a lag activation function. For instance, the Doppler intensity variations from ten Doppler frames before the point of pressure release and up until one frame after the point of pressure release can be used when computing the correlation. This results in a correlation value for each pixel in the Doppler image showing its consistency with the expected hyperemic response, together constituting a correlation map after pressure release. The correlation maps therefore depict the distribution of pixels (or voxels) where Doppler signal intensities at the point of pressure release (i.e., deflate) and follow the pattern of the activation function (e.g., a rapid increase signifying an immediate hyperemic response).

The correlation map(s) can then be binarized and used to mask the Doppler image frames. For instance, the correlation map(s) can be binarized using a threshold value, such that pixels values in the correlation map above the threshold are assigned one binary value (e.g., 1) and pixel values below the threshold are assigned a different binary value (e.g., 0). As a non-limiting example, the threshold can be set to 0.5 to strike a balance between the inclusion of slow responding flow elements (such as muscle perfusion signals) and the inclusion of noise. The binarized images depict all the pixels that exhibited a compensatory flow response, including muscle perfusion. These binarized images can be referred to as correlation masks.

The correlation masks are used to monitor Doppler intensity alterations by averaging the masked Doppler signal magnitude as a function of Doppler frame sample time for the duration of the study. These temporal intensity profiles can then be used to estimate post-occlusion Doppler intensity variations with respect to the baseline.

By defining a multiple (e.g., 4) frame lag activation function the delay in post-occlusion response can be evaluated. By cross-correlating the Doppler intensity variations with shifted versions of this activation function and finding the shifts that result in maximum correlations, it can be determined how many post-occlusion frames it takes for each pixel to exhibit a compensatory flow response.

Lag image data are then generated, as indicated at step 108. As an example, the lag images can be generated by computing lagged correlation maps through cross-correlation, indicating response times for different pixels. The correlation of pixel intensity variations can be computed with lagged activation functions (e.g., up to 4 different lags). The lagged correlation maps generated by computing the correlation between the Doppler image frames and the lagged activation functions result in lag images, which indicate the response delay in each pixel.

Maximum correlation map data are also generated, as indicated at step 110. Creating maximum correlation maps, finding which pixels show correlation above a defined threshold, thereby determining the responding region. By cross-correlating the Doppler intensity variations with shifted versions of the activation function (i.e., the lagged activation functions) and finding the shifts that result in maximum correlations, it can be determined how many post-occlusion frames it takes for each pixel to exhibit a compensatory flow response. The resulting maximum correlation maps indicate all of the pixels that exhibit a hyperemic response.

The maximum correlation maps can be binarized and used to create a region of post-compression response (i.e., a total response region). The maximum correlation maps can be binarized using a thresholding technique. The threshold may be an empirically determined threshold value. As a non-limiting example, a threshold of 0.6 can be used.

Flow variation metric data are then generated from the correlation maps, lag images, and/or maximum correlation maps, as indicated at step 112. Collectively, the correlation maps, lag images, and maximum correlation maps may be referred to as correlation map data that are generated from the Doppler image frames based on the correlation with one or more activation functions, which may include lagged activation functions as described above.

As a non-limiting example, one or more of the following four different flow variation metrics can be computed: a post-occlusion to baseline flow intensity variation (PBFIV), a total response region (TRR), a lag-zero response region (LORR), and a lag-four (and more) response region (L4+RR). PBFIV quantifies the relative increase in blood flow in response to cuff occlusion, TRR approximates the density of the pixels that exhibit a hyperemic response within a few frames post-occlusion, LORR represents the relative size of the region where the quickest measurable response to cuff occlusion occurs, and L4+RR shows the extent of the region where the hyperemic response (if any) takes at least 4 Doppler frames to appear.

The PBFIV metric can be computed as the percentage-wise ratio of the difference between average post-occlusion and average baseline flow intensities over the average baseline intensity as:

$$PBFIV ( \% ) = \frac{{I}_{Post - occ - 5 {frames}_{mean}} - {I}_{{Baseline}_{mean}}}{{I}_{{Baseline}_{mean}}} \times 100 ; \qquad ( 1 )$$

where I_{post-occ-5frames}_{ₘₑₐₙ} is the mean Doppler intensity in the first five post-occlusion Doppler frames after pressure release, and I_{Baseline}_{ₘₑₐₙ} is the baseline mean Doppler intensity. The PBFIV metric can be computed using the correlation map data and/or correlation masked Doppler image frames described above.

The TRR metric can be computed as the imaging area where maximum correlations in the corresponding maximum correlation map exceed a predefined threshold:

$$TRR ( \% ) = \frac{{N}_{MaxCorr}}{{A}_{Image}} \times 100 ; \qquad ( 2 )$$

where N_{MaxCorr} is the pixel count in the maximum correlation map after binarization, and A_{Image} is the total number of pixels in the maximum correlation map.

The Lag0RR metric can be computed as the area in which flow increase happens immediately after pressure release:

$$L 0 RR ( \% ) = \frac{{N}_{Lag 0}}{{A}_{Image}} \times 100 ; \qquad ( 3 )$$

where N_{Lag0} is the number of pixels corresponding to Lag0 (high correlation with a zero lag activation function), and A_{Image} is the total number of pixels in the lag images.

The Lag4+RR metric can be defined as the area in which flow increase after pressure release takes at least four Doppler frames to manifest:

$$L 4 + RR ( \% ) = \frac{{N}_{Lag 4 +}}{{A}_{Image}} \times 100 ; \qquad ( 4 )$$

where N_{Lag4+} is the number of pixels corresponding to Lag4 (and more), and A_{Image} is the total number of pixels in the lag images.

The flow variation metrics can then be displayed to a user, stored for later use or further processing, or both, as indicated at step 114. As one example, the flow variation metrics can be output by the computer system by generating a report based on the flow variation metrics. The report may include textual and/or quantitative numerical data. Additionally or alternatively, the report may include image data. For instance, the report may include combining the ultrasound data, Doppler image frames, correlation maps, lag image, maximum correlation maps, or other images with the flow variation metrics and displaying the combined data to a user via the computer system.

In some implementations, the flow variation metrics can be used as an input to a classification algorithm to generate classified feature data. The classification algorithm may include a machine learning model trained on training data to generate classified feature data from an input of one or more flow variation metrics. The machine learning model may include a neural network. Additionally or alternatively, the machine learning model may include random forest model, support vector machine model, a naïve Bayes classifier, a nearest neighbors model, a decision tree model, an adaptive boosting (AdaBoost) model, a quadratic discriminant analysis (QDA) model, a Gaussian process model, or the like.

The classified feature data may include a risk score. The risk score can provide physicians or other clinicians with a recommendation to consider additional monitoring for subjects whose flow variation metrics indicate the likelihood of the subject suffering from a particular medical condition.

As another example, the classified feature data may indicate the probability for a particular classification (i.e., the probability that the flow variation metrics include patterns, features, or characteristics indicative of detecting, differentiating, and/or determining the severity of one or more medical conditions).

Additionally or alternatively, the classified feature data may classify the flow variation metrics as indicating a particular medical condition. In these instances, the classified feature data can differentiate between different medical conditions. In still other embodiments, the classified feature data may indicate a severity of a medical condition. For example, the classified feature data may include a severity score that quantifies a severity of a medical condition.

In an example study, the disclosed systems and methods were implemented to analyze the blood flow variations in a group of 14 patients with clinical diagnosis of PAD (7 male, 7 female), and 8 healthy volunteers (1 male, 7 female). Age distribution of the patients and healthy subjects was 65.9+16.3 and 64.1+2.8, respectively (mean±standard deviation). During the study, both legs of the subjects were scanned. For final data analysis, data from legs of patients with normal ABI, as well as poor data acquisitions were excluded. After exclusions, data from a total of 13 legs with abnormal ABI were compared to data from 13 legs of healthy individuals.

Data acquisition was performed using a linear array L11-4v probe (with a center frequency of 7.24 MHz) attached to a Verasonics Vantage 256 ultrasound research system (Verasonics Inc., Kirkland, WA, USA). Ultrafast ultrasound imaging was implemented through coherent compounding of plane wave transmissions at five different insonification angles equally spaced within the [−5.5°+5.5°] range. Each transmission sequence involved acquiring 500 IQ data frames at a frame rate of 2 kHz and was repeated every 2 s to monitor the flow response over time.

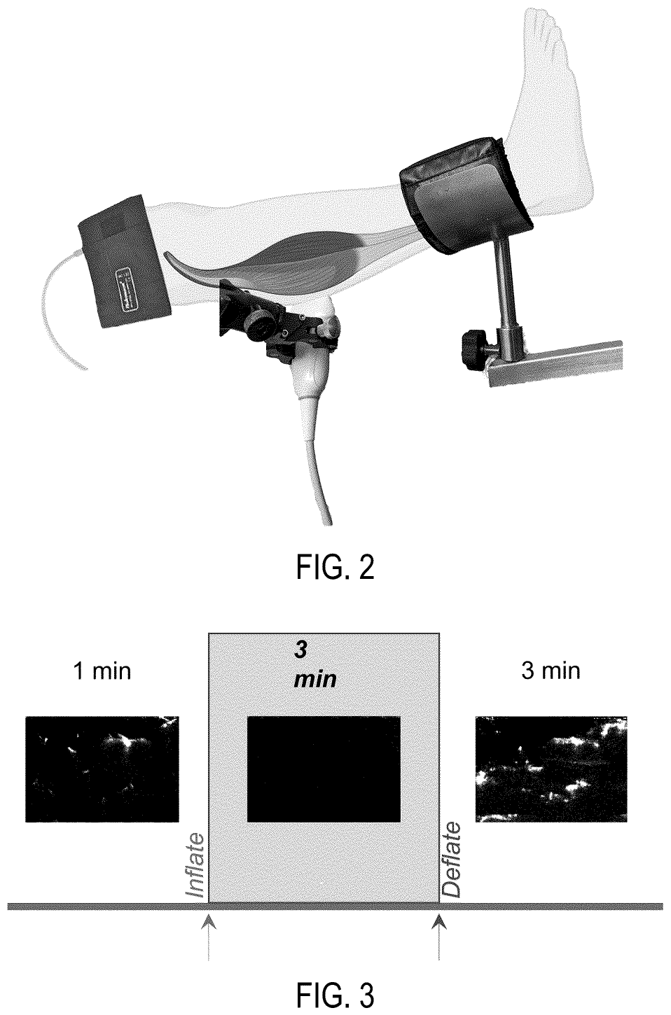

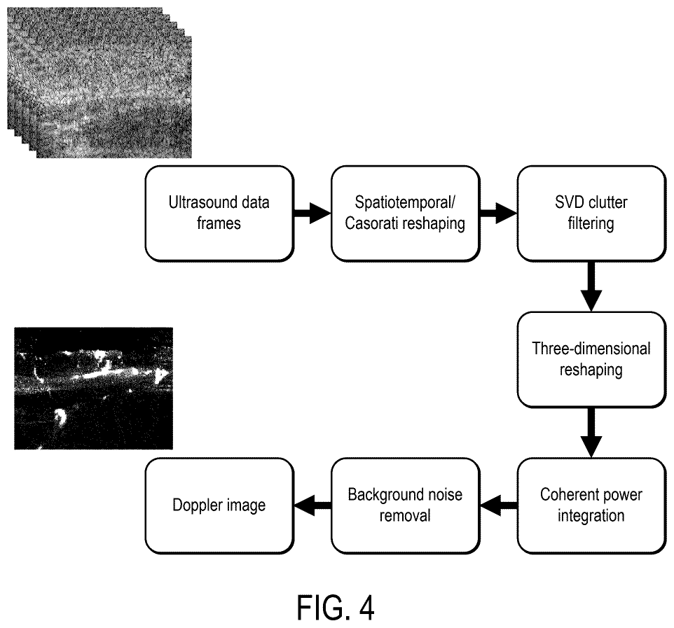

With subjects in the supine position, the targeted leg was placed on a stand-alone leg prepper. The ultrasound probe was attached to the subject's calf muscle for imaging and was secured in place using a multi-joint mechanical arm. A pressure cuff was wrapped around the subject's thigh and an automatic cuff inflation device (D. E. Hokanson Inc., Bellevue, WA, USA) was used for rapid inflation of the cuff. A schematic illustration of the setup is shown in FIG. 2. Ultrasound data was continuously collected for 7 minutes per each leg. The setup included one minute of baseline data acquisition, followed by three minutes of pressure-induced occlusion and three minutes of post-occlusion data acquisition. FIG. 3 depicts a schematic of the timeline of the acquisition setup, as well as examples of Doppler images corresponding to each portion of the study.

The 500 IQ data frames acquired during each transmission sequence constitute an ensemble of ultrasound images that was used to generate a Doppler image visualizing blood flow intensities at a given point in time. These frames were first reshaped into a spatiotemporal/Casorati data matrix. Next, a singular value decomposition (SVD) filter was applied to the data to remove the low-rank tissue clutter. In this study, the SVD threshold for the separation of clutter and blood subspaces was chosen empirically and was set to the constant value of 50 for all the Doppler frames (no upper threshold for noise removal). Subsequently, the clutter filtered data frame ensemble underwent a temporal coherent integration to generate the Doppler image.

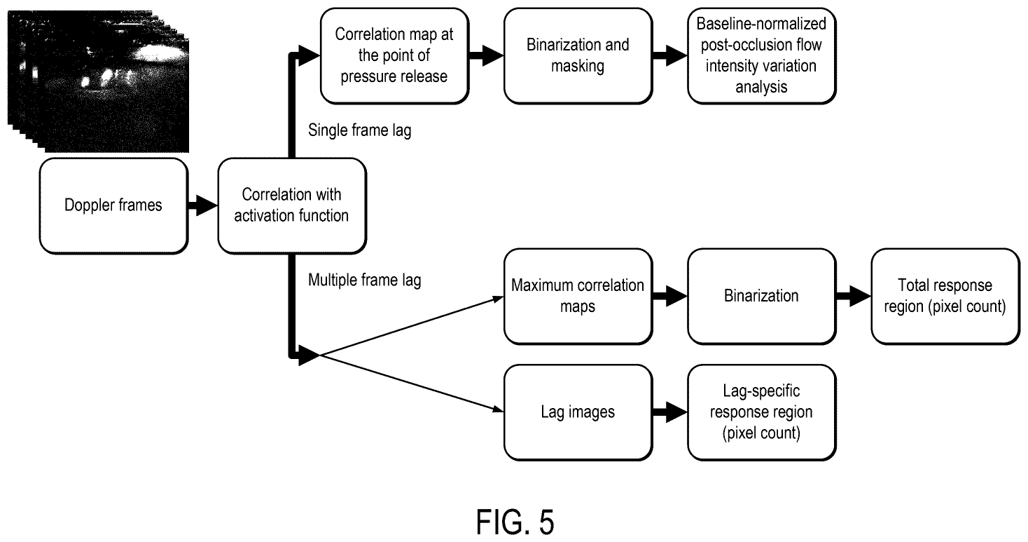

The resulting Doppler image was composed of signals generated by blood flow as well as noise. The background noise profile (mainly induced by the time gain compensation (TGC) settings) can be estimated and compensated for, through various means. In this example study, a Doppler image generated from an open-air transmission was used to estimate this profile. Since no significant echo signal was expected in such a transmission, the resulting image would be a good approximation of the TGC-induced noise pattern. Considering the TGC-induced noise was removed after the implementation of SVD, it did not have a direct influence on our choice of the SVD threshold. The noise profile was then subtracted from each of the Doppler images to generate a final flow image. FIG. 4 illustrates an example workflow of different stages of generating these Doppler frames.

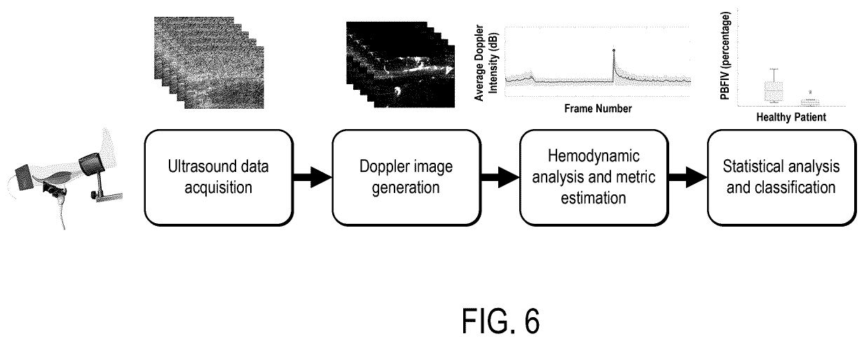

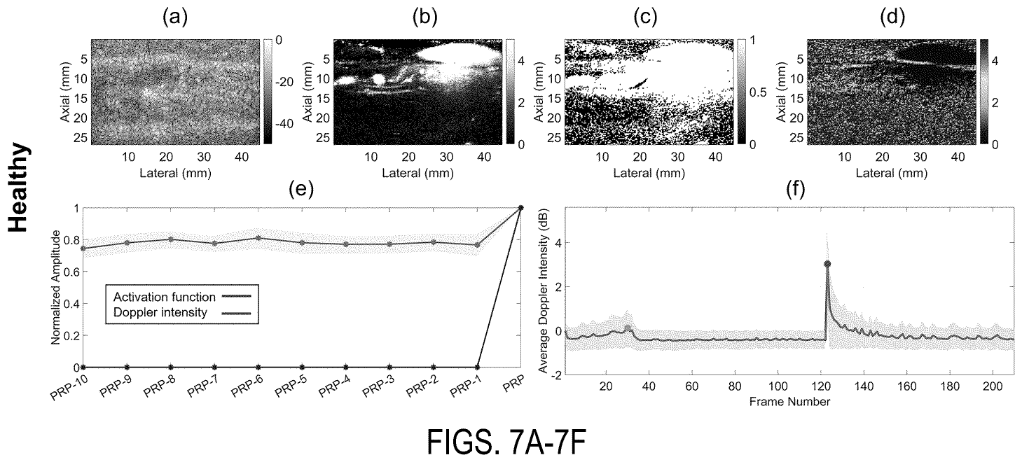

Quantitative flow variation metrics were then computed using the methods described in the present disclosure. FIG. 5 shows different stages of this process. FIG. 6 depicts a flowchart of the entire method, from data acquisition to metric estimation for potential diagnostic applications.

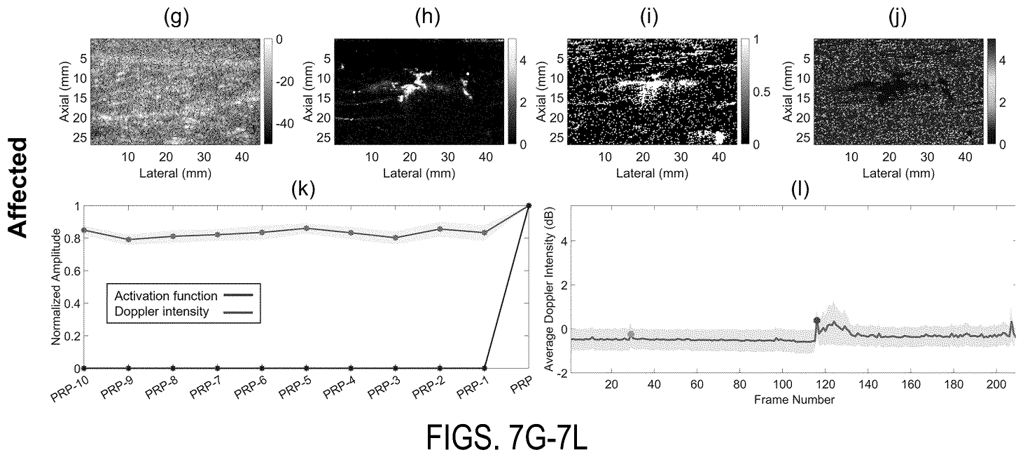

Comparative illustration of the results for the aforementioned cases is displayed in FIG. 7. FIGS. 7A and 7G show example B-mode images of scanned area. Obtained Doppler frames after the pressure release point (PRP) are shown in FIGS. 7B and 7H. Binarized correlation masks are presented in FIGS. 7C and 7I. These masks show pixels at which temporal Doppler signals have a correlation larger than 0.5 with the single frame lag activation function. Comparing the two figures, fewer pixels demonstrate a correlated behavior with the activation function (i.e., a rapid hyperemic response to pressure release) in the case of the PAD patient. FIGS. 7D and 7J illustrate the lag images for the healthy and affected legs, respectively. Dark blue regions represent lag0 (no lag) pixels where an immediate surge of flow occurs. A larger region is covered by such pixels in FIG. 7D compared to FIG. 7J. FIGS. 7E and 7K show the average of the normalized temporal Doppler signals (red line) for all pixels within the correlation masks, as well as the single frame lag activation function (blue line), for 10 frames before PRP up to PRP, exhibiting a sharp increase in the amplitude of the signal at the time of pressure release in correlation with the activation function. The shaded area around the red line in light blue shows half of the standard deviation of variations for all pixels on each side of the line. FIGS. 7F and 7L depict the average of the Doppler intensities of the pixels inside the correlation masks at each Doppler frame. These variations demonstrate the hemodynamic response to cuff inflation (green circles) and deflation (red circles). The inflation and deflation frames are chosen as the Doppler frames that are closest in time to the actual inflation and deflation events during the study. The general trend in the responses indicates a decline in flow after inflation and a rise and gradual fall after deflation. A more intense and rapid response to pressure release is observed in FIG. 7F compared to FIG. 7L.

A summary of the calculated metric values is presented in Table 1.

TABLE 1
Hemodynamic response metrics and their corresponding p-values (metrics
distributions are presented as mean ± standard deviation).

| Metrics | PAD | Healthy | P-value |
|---|---|---|---|
| PBFIV* (percentage) | 123.02 ± 149.30 | 672.86 ± 800.94 | 0.0015 |
| TRR** (percentage) | 3.61 ± 6.42 | 7.42 ± 6.50 | 0.0183 |
| L0RR*** (percentage) | 22.60 ± 11.86 | 37.19 ± 14.89 | 0.0048 |
| L4 + RR**** | 1.99 ± 1.63 | 1.61 ± 1.22 | 0.7196 |
| (percentage) | | | |

*PBFIV: Post-occlusion to baseline flow intensity variation.
**TRR: Total response region.
***L0RR: Lag0 response region.
****L4 + RR: Lag4 (and more) response region.

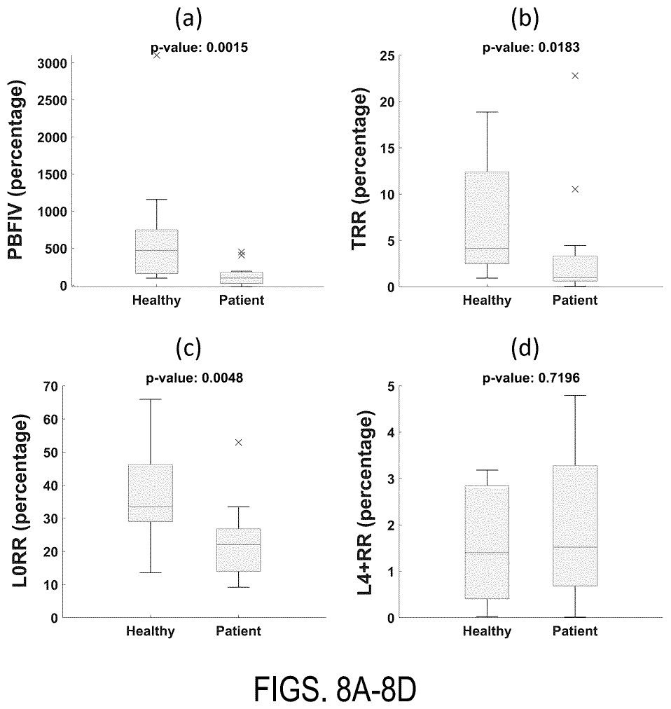

The corresponding box-and-whisker plots of the distributions of the metrics for the two groups are also illustrated in FIGS. 8A-8D. The post-occlusion to baseline flow intensity variations (PBFIV) show the net increase in Doppler intensity with respect to the average baseline Doppler intensity. This parameter shows an average of 672 percent increase in Doppler intensity in response to pressure release for the healthy leg compared to 123 percent for the affected legs. Total response region (TRR) represents the percentage of the pixels that have a higher than 60 percent correlation with at least one shifted version of the multiple frame lag activation function. This region constitutes an average of about a 3.61 percent of the entire scanning region for the PAD cases compared to 7.42 percent for the healthy group. Finally, based on the lag images, in the case of affected legs, for nearly 1.99 percent of the scanned area on average, it takes at least four (or more) frames to manifest a hyperemic response (if they do so at all), while this value is about 1.61 percent for the healthy subjects (L4+RR). On the other hand, an average of about 37.19 percent of the region demonstrates an immediate (lag 0) flow compensation in the case of healthy legs compared to 22.60 percent in the case of affected legs. In total, three out of the four utilized metrics exhibited a significant (p-value >0.05) distributional difference between the two groups.

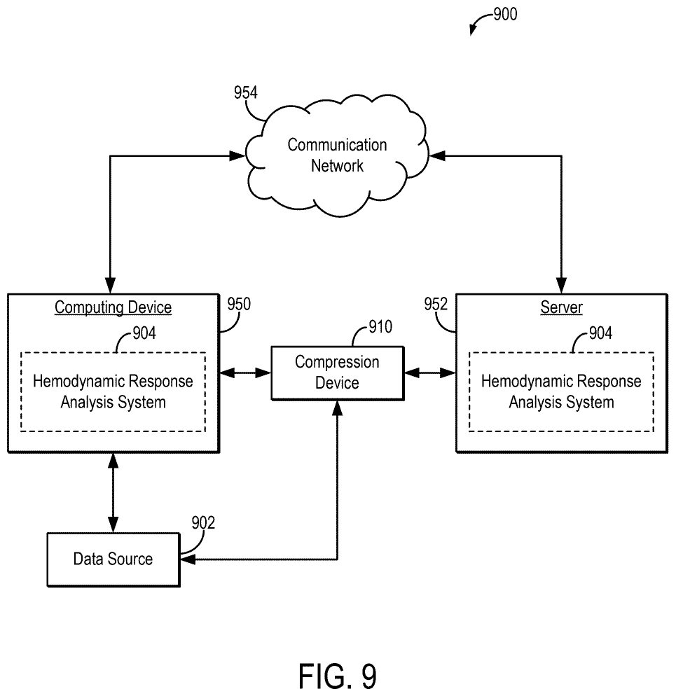

FIG. 9 shows an example of a system 900 for hemodynamic response analysis in accordance with some embodiments described in the present disclosure. As shown in FIG. 9, a computing device 950 can receive one or more types of data (e.g., ultrasound data) from data source 902. In some embodiments, computing device 950 can execute at least a portion of a hemodynamic response analysis system 904 to generate and/or analyze flow variation metrics from data received from the data source 902.

Additionally or alternatively, in some embodiments, the computing device 950 can communicate information about data received from the data source 902 to a server 952 over a communication network 954, which can execute at least a portion of the hemodynamic response analysis system 904. In such embodiments, the server 952 can return information to the computing device 950 (and/or any other suitable computing device) indicative of an output of the hemodynamic response analysis system 904.

In some embodiments, computing device 950 and/or server 952 can be any suitable computing device or combination of devices, such as a desktop computer, a laptop computer, a smartphone, a tablet computer, a wearable computer, a server computer, a virtual machine being executed by a physical computing device, and so on. The computing device 950 and/or server 952 can also reconstruct images from the data, process such images, etc. The computing device 950 and/or server 952 can also control the operation of a compression device 910 (e.g., a pressure cuff or other compression device) to provide compression to the lower extremity of a subject. The pressure cuff or other compression device can be operated to synchronize data acquisition with an ultrasound system to a prescribed data acquisition procedure in which data are acquired before, during, and after compression is applied to the lower extremity.

In some embodiments, data source 902 can be any suitable source of data (e.g., measurement data, images reconstructed from measurement data, processed image data), such as an ultrasound system, another computing device (e.g., a server storing measurement data, images reconstructed from measurement data, processed image data), and so on. In some embodiments, data source 902 can be local to computing device 950. For example, data source 902 can be incorporated with computing device 950 (e.g., computing device 950 can be configured as part of a device for measuring, recording, estimating, acquiring, or otherwise collecting or storing data). As another example, data source 902 can be connected to computing device 950 by a cable, a direct wireless link, and so on. Additionally or alternatively, in some embodiments, data source 902 can be located locally and/or remotely from computing device 950, and can communicate data to computing device 950 (and/or server 952) via a communication network (e.g., communication network 954).

In some embodiments, communication network 954 can be any suitable communication network or combination of communication networks. For example, communication network 954 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 954 can be a local area network, a wide area network, a public network (e.g., the Internet), a private or semi-private network (e.g., a corporate or university intranet), any other suitable type of network, or any suitable combination of networks. Communications links shown in FIG. 9 can each be any suitable communications link or combination of communications links, such as wired links, fiber optic links, Wi-Fi links, Bluetooth links, cellular links, and so on.

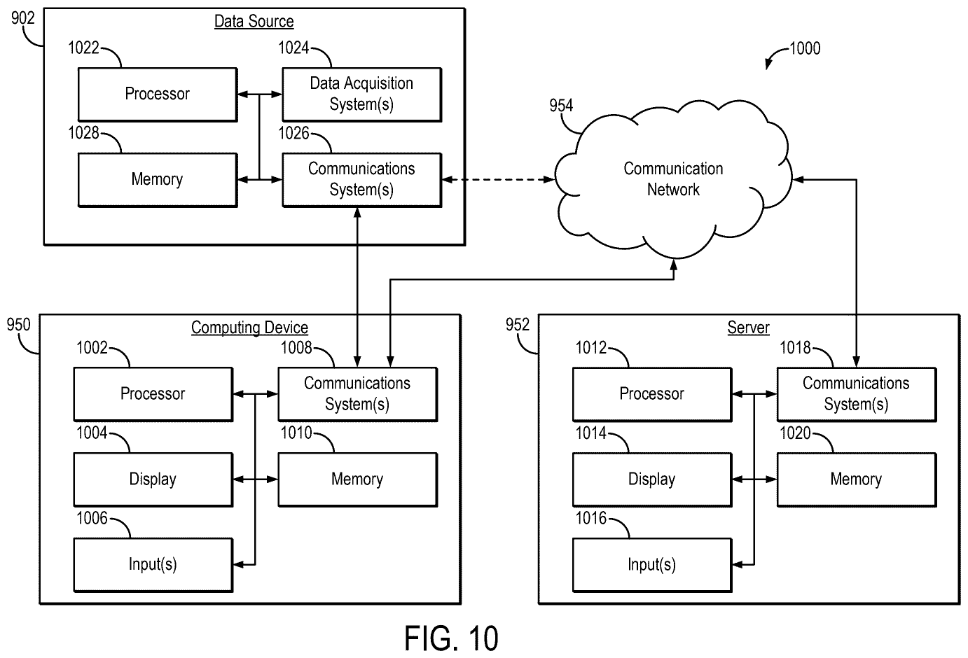

Referring now to FIG. 10, an example of hardware 1000 that can be used to implement data source 902, computing device 950, and server 952 in accordance with some embodiments of the systems and methods described in the present disclosure is shown.

As shown in FIG. 10, in some embodiments, computing device 950 can include a processor 1002, a display 1004, one or more inputs 1006, one or more communication systems 1008, and/or memory 1010. In some embodiments, processor 1002 can be any suitable hardware processor or combination of processors, such as a central processing unit (“CPU”), a graphics processing unit (“GPU”), and so on. In some embodiments, display 1004 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1006 can include any suitable input devices and/or sensors that can be used to receive user input, such as a keyboard, a mouse, a touchscreen, a microphone, and so on.

In some embodiments, communications systems 1008 can include any suitable hardware, firmware, and/or software for communicating information over communication network 954 and/or any other suitable communication networks. For example, communications systems 1008 can include one or more transceivers, one or more communication chips and/or chip sets, and so on. In a more particular example, communications systems 1008 can include hardware, firmware, and/or software that can be used to establish a Wi-Fi connection, a Bluetooth connection, a cellular connection, an Ethernet connection, and so on.

In some embodiments, memory 1010 can include any suitable storage device or devices that can be used to store instructions, values, data, or the like, that can be used, for example, by processor 1002 to present content using display 1004, to communicate with server 952 via communications system(s) 1008, and so on. Memory 1010 can include any suitable volatile memory, non-volatile memory, storage, or any suitable combination thereof. For example, memory 1010 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1010 can have encoded thereon, or otherwise stored therein, a computer program for controlling operation of computing device 950. In such embodiments, processor 1002 can execute at least a portion of the computer program to present content (e.g., images, user interfaces, graphics, tables), receive content from server 952, transmit information to server 952, and so on. For example, the processor 1002 and the memory 1010 can be configured to perform the methods described herein (e.g., the method of FIG. 1; the workflow illustrated in FIG. 4; the workflow illustrated in FIG. 5; the workflow illustrated in FIG. 6).

In some embodiments, server 952 can include a processor 1012, a display 1014, one or more inputs 1016, one or more communications systems 1018, and/or memory 1020. In some embodiments, processor 1012 can be any suitable hardware processor or combination of processors, such as a CPU, a GPU, and so on. In some embodiments, display 1014 can include any suitable display devices, such as an LCD screen, LED display, OLED display, electrophoretic display, a computer monitor, a touchscreen, a television, and so on. In some embodiments, inputs 1016 can include any suitable input devices and/or sensors that can be used to receive user input, such as a keyboard, a mouse, a touchscreen, a microphone, and so on.

In some embodiments, communications systems 1018 can include any suitable hardware, firmware, and/or software for communicating information over communication network 954 and/or any other suitable communication networks. For example, communications systems 1018 can include one or more transceivers, one or more communication chips and/or chip sets, and so on. In a more particular example, communications systems 1018 can include hardware, firmware, and/or software that can be used to establish a Wi-Fi connection, a Bluetooth connection, a cellular connection, an Ethernet connection, and so on.

In some embodiments, memory 1020 can include any suitable storage device or devices that can be used to store instructions, values, data, or the like, that can be used, for example, by processor 1012 to present content using display 1014, to communicate with one or more computing devices 950, and so on. Memory 1020 can include any suitable volatile memory, non-volatile memory, storage, or any suitable combination thereof. For example, memory 1020 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1020 can have encoded thereon a server program for controlling operation of server 952. In such embodiments, processor 1012 can execute at least a portion of the server program to transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone), and so on.

In some embodiments, the server 952 is configured to perform the methods described in the present disclosure. For example, the processor 1012 and memory 1020 can be configured to perform the methods described herein (e.g., the method of FIG. 1; the workflow illustrated in FIG. 4; the workflow illustrated in FIG. 5; the workflow illustrated in FIG. 6).

In some embodiments, data source 902 can include a processor 1022, one or more data acquisition systems 1024, one or more communications systems 1026, and/or memory 1028. In some embodiments, processor 1022 can be any suitable hardware processor or combination of processors, such as a CPU, a GPU, and so on. In some embodiments, the one or more data acquisition systems 1024 are generally configured to acquire data, images, or both, and can include an ultrasound system. Additionally or alternatively, in some embodiments, the one or more data acquisition systems 1024 can include any suitable hardware, firmware, and/or software for coupling to and/or controlling operations of an ultrasound system. In some embodiments, one or more portions of the data acquisition system(s) 1024 can be removable and/or replaceable.

Note that, although not shown, data source 902 can include any suitable inputs and/or outputs. For example, data source 902 can include input devices and/or sensors that can be used to receive user input, such as a keyboard, a mouse, a touchscreen, a microphone, a trackpad, a trackball, and so on. As another example, data source 902 can include any suitable display devices, such as an LCD screen, an LED display, an OLED display, an electrophoretic display, a computer monitor, a touchscreen, a television, etc., one or more speakers, and so on.

In some embodiments, communications systems 1026 can include any suitable hardware, firmware, and/or software for communicating information to computing device 950 (and, in some embodiments, over communication network 954 and/or any other suitable communication networks). For example, communications systems 1026 can include one or more transceivers, one or more communication chips and/or chip sets, and so on. In a more particular example, communications systems 1026 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1028 can include any suitable storage device or devices that can be used to store instructions, values, data, or the like, that can be used, for example, by processor 1022 to control the one or more data acquisition systems 1024, and/or receive data from the one or more data acquisition systems 1024; to generate images from data; present content (e.g., data, images, a user interface) using a display; communicate with one or more computing devices 950; and so on. Memory 1028 can include any suitable volatile memory, non-volatile memory, storage, or any suitable combination thereof. For example, memory 1028 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1028 can have encoded thereon, or otherwise stored therein, a program for controlling operation of data source 902. In such embodiments, processor 1022 can execute at least a portion of the program to generate images, transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

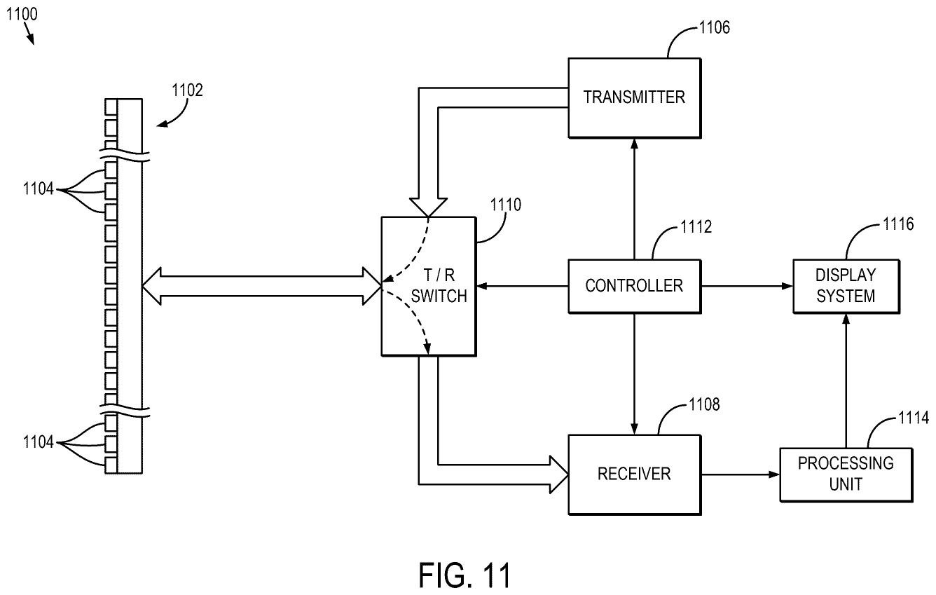

FIG. 11 illustrates an example of an ultrasound system 1100 that can implement the methods described in the present disclosure. The ultrasound system 1100 includes a transducer array 1102 that includes a plurality of separately driven transducer elements 1104. The transducer array 1102 can include any suitable ultrasound transducer array, including linear arrays, curved arrays, phased arrays, and so on. Similarly, the transducer array 1102 can include a 1D transducer, a 1.5D transducer, a 1.75D transducer, a 2D transducer, a 3D transducer, and so on.

When energized by a transmitter 1106, a given transducer element 1104 produces a burst of ultrasonic energy. The ultrasonic energy reflected back to the transducer array 1102 (e.g., an echo) from the object or subject under study is converted to an electrical signal (e.g., an echo signal) by each transducer element 1104 and can be applied separately to a receiver 1108 through a set of switches 1110. The transmitter 1106, receiver 1108, and switches 1110 are operated under the control of a controller 1112, which may include one or more processors. As one example, the controller 1112 can include a computer system.

The transmitter 1106 can be programmed to transmit unfocused or focused ultrasound waves. In some configurations, the transmitter 1106 can also be programmed to transmit diverged waves, spherical waves, cylindrical waves, plane waves, or combinations thereof. Furthermore, the transmitter 1106 can be programmed to transmit spatially or temporally encoded pulses.

The receiver 1108 can be programmed to implement a suitable detection sequence for the imaging task at hand. In some embodiments, the detection sequence can include one or more of line-by-line scanning, compounding plane wave imaging, synthetic aperture imaging, and compounding diverging beam imaging.

In some configurations, the transmitter 1106 and the receiver 1108 can be programmed to implement a high frame rate. For instance, a frame rate associated with an acquisition pulse repetition frequency (“PRF”) of at least 100 Hz can be implemented. In some configurations, the ultrasound system 1100 can sample and store at least one hundred ensembles of echo signals in the temporal direction.

The controller 1112 can be programmed to implement an imaging sequence using the techniques described in the present disclosure. For instance, the controller 1112 can control operation of the transducer array 1102 to transmit ultrasound and receive echo signals in connection with applying a pressure or compression to a lower extremity of a subject.

A scan can be performed by setting the switches 1110 to their transmit position, thereby directing the transmitter 1106 to be turned on momentarily to energize transducer elements 1104 during a single transmission event according to the imaging sequence. The switches 1110 can then be set to their receive position and the subsequent echo signals produced by the transducer elements 1104 in response to one or more detected echoes are measured and applied to the receiver 1108. The separate echo signals from the transducer elements 1104 can be combined in the receiver 1108 to produce a single echo signal.

The echo signals are communicated to a processing unit 1114, which may be implemented by a hardware processor and memory, to process echo signals or images generated from echo signals. As an example, the processing unit 1114 can generate Doppler image frames, correlation map data, and flow variation metric data using the methods described in the present disclosure. Images produced from the echo signals by the processing unit 1114 can be displayed on a display system 1116.

The present disclosure has described one or more preferred embodiments, and it should be appreciated that many equivalents, alternatives, variations, and modifications, aside from those expressly stated, are possible and within the scope of the invention.

1. A method for generating quantitative flow variation metrics from non-contrast ultrasound data, the steps of the method comprising:
(a) providing ultrasound data to a computer system, the ultrasound data having been acquired with an ultrasound system from a lower extremity of a subject during:
a first duration of time during which no compression is applied to the lower extremity;
a second duration of time during which compression is applied to the lower extremity, wherein the second duration of time occurs after the first duration of time;
a third duration of time during which no compression is applied to the lower extremity, wherein the third duration of time occurs after the second duration of time;
(b) generating a series of Doppler image frames from the ultrasound data using the computer system, wherein the Doppler image frames depict perfusion in the lower extremity of the subject;
(c) generating correlation map data with the computer system by correlating the series of Doppler image frames with at least one activation function that models the compression applied to the lower extremity;
(d) generating flow variation metric data from the correlation map data using the computer system; and
(e) outputting the flow variation metric data using the computer system.

2. The method of claim 1, wherein the correlation map data comprise correlation maps generated by correlating the series of Doppler image frames with the activation function, wherein the at least one activation function comprises zero lag relative to the compression applied to the lower extremity.

3. The method of claim 2, wherein the flow variation metric data comprise a post-occlusion to baseline flow intensity variation (PBFIV) metric computed by:
generating masked Doppler image frames by masking the Doppler image frames using the correlation maps;
computing an average post-occlusion flow intensity by averaging a plurality of the masked Doppler image frames associated with ultrasound data acquired during the third duration of time;
computing an average baseline flow intensity by averaging a plurality of the masked Doppler image frames associated with ultrasound data acquired during the first duration of time;
computing a flow intensity difference as a difference of the average post-occlusion flow intensity and the average baseline flow intensity; and
computing a ratio of the flow intensity difference and the average baseline flow intensity;
wherein the PBFIV indicates Doppler intensity variations following the compression of the lower extremity relative to a baseline flow.

4. The method of claim 3, wherein masking the Doppler image frames comprises generating a correlation mask for each Doppler image frame by binarizing a corresponding correlation map and multiplying the correlation mask with the Doppler image frame.

5. The method of claim 4, wherein the correlation mask is generated by thresholding the corresponding correlation map using a threshold.

6. The method of claim 5, wherein the threshold is 0.5.

7. The method of claim 1, wherein the correlation map data comprise lag images generated by correlating the series of Doppler image frames with lagged activation functions, wherein the lagged activation functions comprise a plurality of different temporal lags relative to the compression applied to the lower extremity.

8. The method of claim 7, wherein the flow variation metric data comprise a lag-zero response region (LORR) metric computed as a ratio between a number of pixels with zero lag and a total number of pixels in a lag image, wherein the LORR indicates a density of pixels exhibiting an immediate increase in flow following pressure release of the compression.

9. The method of claim 7, wherein the flow variation metric data comprise a lag-four plus response region (L4+RR) metric computed as a ratio between a number of pixels with at least four frames of lag and a total number of pixels in a lag image, wherein the L4+RR indicates pixels for which at least four frames are required to manifest a compensatory response.

10. The method of claim 7, wherein the correlation map data comprise maximum correlation maps generated by identifying maximum correlation values in the lag images and storing the maximum correlation values as the maximum correlation maps.

11. The method of claim 10, wherein the flow variation metric data comprise a total response region (TRR) metric computed by:
generating binarized maximum correlation maps by thresholding the maximum correlation maps with a threshold; and
computing, for each maximum correlation map and corresponding binarized maximum correlation map, a ratio between a number of nonzero pixels in the binarized maximum correlation map and a total number of pixels in the maximum correlation map, wherein the TRR indicates a region of post-compression response in the lower extremity of the subject.

12. The method of claim 11, wherein the threshold is 0.6.

13. The method of claim 1, further comprising inputting the flow variation metric data to a classification algorithm, generating classified feature data as an output.

14. The method of claim 13, wherein the classification algorithm comprises a machine learning model trained on training data comprising flow variation metrics generated from a population of subjects.

15. The method of claim 14, wherein the machine learning model comprises a neural network.

16. The method of claim 14, wherein the machine learning model comprises a decision tree model.

17. The method of claim 13, wherein the classified feature data comprise a risk score indicating a likelihood of the subject suffering from a particular medical condition.

18. The method of claim 13, wherein the classified feature data comprise a probability that the flow variation metric data include at least one of patterns, features, or characteristics indicative of a particular medical condition.

19. The method of claim 1, wherein the ultrasound data are acquired using a high-frame-rate plane-wave ultrasound imaging sequence without using a contrast agent.

20. The method of claim 19, wherein the high-frame-rate plane-wave ultrasound imaging sequence comprises coherent compounding of plane wave transmissions at a plurality of different insonification angles.

21. A method for assessing blood flow in a lower extremity using ultrasound, the method comprising:
acquiring ultrasound data from the lower extremity of a subject wearing a pressure cuff around a portion of their lower extremity while the pressure cuff is applying a compression to the lower extremity;
generating one or more Doppler image frames from the ultrasound data;
generating correlation map data from the Doppler image frames based on correlation with one or more activation functions that model the compression applied to the lower extremity by the pressure cuff; and
generating flow variation metric data from the correlation map data, wherein the flow variation metric data provide a quantitative assessment of blood flow in the lower extremity.

22. The method of claim 21, wherein generating the correlation map data comprises:
generating one or more correlation maps by correlating temporal Doppler signal intensity variations in the ultrasound data with a lag activation function; and
generating one or more lag images by computing lagged correlation maps through cross-correlation.

23. The method of claim 22, further comprising generating maximum correlation map data by cross-correlating Doppler intensity variations with shifted versions of the lag activation function.

24. The method of claim 21, wherein acquiring the ultrasound data comprises acquiring the ultrasound data using high-frame-rate plane-wave ultrasound microvessel imaging without using contrast agents.

25. The method of claim 24, wherein the ultrasound data are acquired using an ultrasound system implementing coherent compounding of plane wave transmissions at multiple different insonification angles.

26. The method of claim 25, wherein the multiple different insonification angles comprise five different insonification angles equally spaced within a range of −5.5 degrees to +5.5 degrees.

27. A method for generating flow variation metrics from ultrasound data acquired from a lower extremity of a subject, the method comprising:
acquiring ultrasound data from the lower extremity of the subject wearing a pressure cuff around a portion of the lower extremity;
generating one or more Doppler image frames from the ultrasound data;
generating correlation maps from the Doppler image frames using a lag activation function;
generating lag image data from the Doppler image frames;
generating maximum correlation map data from the Doppler image frames; and
generating flow variation metric data from the correlation maps, lag image data, and maximum correlation map data.

28. The method of claim 27, wherein generating the correlation maps comprises correlating temporal Doppler signal intensity variations in the ultrasound data with the lag activation function; and
binarizing the correlation maps using a threshold value.

29. The method of claim 27, wherein generating the lag image data comprises generating lagged correlation maps through cross-correlation with shifted versions of the lag activation function.

30. The method of claim 29, wherein the shifted versions of the lag activation function include up to 4 different lags.

31. The method of claim 27, wherein generating the maximum correlation map data comprises:
cross-correlating Doppler intensity variations with shifted versions of the lag activation function; and
finding shifts that result in maximum correlations.

32. The method of claim 27, wherein generating the flow variation metric data comprises computing at least one of a post-occlusion to baseline flow intensity variation (PBFIV) metric, a total response region (TRR) metric, a lag-zero response region (LORR) metric, or a lag-four and more response region (L4+RR) metric.

33. The method of claim 27, further comprising generating classified feature data by inputting the flow variation metric data to a classification algorithm, wherein the classified feature data indicate at least one of a risk score indicating a likelihood of the subject having a particular vascular health condition; a probability value representing a likelihood of the flow variation metric data corresponding to a specific vascular health classification; a categorical classification indicating presence or absence of a vascular abnormality; or a severity score quantifying a degree of vascular impairment in the lower extremity.
